(19) United States
(12) Patent Application Publication
(10) Pub. No.: US 2012/0236987 A1
(43) Pub. Date: Sep. 20, 2012

(54) MULTIPLE ENERGY CT SCANNER

(76) Inventors: DAVID RUIMI, GANOT HADAR (IL); OLGA SHAPIRO, HAIFA (IL); EHUD DAFNI, CAESAREA (IL)

(21) Appl. No.: 13/422,099

(22) Filed: Mar. 16, 2012

Related U.S. Application Data
(60) Provisional application No. 61/465,358, filed on Mar. 18, 2011.

(51) Int. Cl. A61B 6/03; G01N 23/04

(52) U.S. Cl. 378/19

(57) ABSTRACT

A CT scanner for multiple energy CT scanning of a subject having an X-Ray source adapted to rotate about the subject; and a detector array, having a plurality of detector elements, adapted to acquire attenuation data for X-Rays that have been attenuated by a subject disposed between said X-Ray source and said detector array, said detector array comprising at least two types of detector element, which are differ by their spectral response. The scanner is adapted to generate images associated with the different X-Ray energy spectra.

The present invention claims priority from a U.S. Provisional Patent Application filed by Ruimi David et al. on the 18 of Mar. 2011. The application was assigned the Ser. No. 61/465,358.

### FIELD OF THE INVENTION

The present invention relates to Computerized Tomography (CT) imaging and more particularly to multiple energy CT imaging.

### BACKGROUND OF THE INVENTION

Computerized Tomography (CT) scanners produce images of a subject by reconstruction of X-Ray attenuation data acquired over multiple view angles. Typically, cross sectional images are constructed by back projecting the view data received from the CT detector over the multiple views. Typically, X-Ray sources of wide energy spectrum are used and the CT images are representation of the energy-averaged X-Ray attenuation coefficient at each image pixel, referred to as a CT number. CT number provides information regarding density of the scanned subject but they do not provide direct information on the material composition of the tissue. For example, in medical imaging, bone tissue comprising calcium may have a similar CT number to a blood vessel filled with iodine based contrast agent.

Dual-energy CT is a known technique, wherein the spectral dependence of the X-Ray attenuation of a subject is measured using X-Ray source (or sources) having two different energy spectra. The different spectral attenuation behavior of a subject under examination is caused by different X-Ray attenuation physical effects like Photoelectric-effect and Compton scattering. Different materials have a different spectral dependence of the attenuation. Thus, dual-energy CT enables an improved characterization of material. For example, by acquiring two data sets, one corresponding to lower energy X-Rays and one corresponding to higher energy X-Rays, it is possible to determine both the density and effective atomic number of material in the scanned subject. Dual energy CT is useful not only in medical imaging but for example also in homeland security applications where certain threatening materials cannot be distinguished from ordinary materials based on CT number only but can he distinguished based on measurement of CT number combined with effective atomic number.

Dual-energy CT is typically implemented by applying different high voltages to the X-Ray source, applying filtering to the X-Ray beam, using detectors which are sensitive to the photon energies or combination of the above.

Certain CT scanners provided by Siemens Medical Solutions comprise two X-Ray sources operating simultaneously, each with a corresponding detector array. In dual energy mode each source is operated by a different high voltage.

Certain CT scanners provided by General Electric Healthcare comprise a high voltage generator capable of fast switching between two different output voltages. In dual energy mode the generator switches between two high voltage values, enabling acquisition of two data sets corresponding to two energy spectra.

U.S. Patents applications 20100220833 entitled “Detector array for spectral CT” and 20080210877 entitled “Double Decker detector for spectral CT”, the content of both is incorporated herein by reference, describe detector arrays for dual energy imaging comprising two layers of detector elements, the upper layers more sensitive to low energy radiation and the lower level more sensitive to high energy radiation.

U.S. Pat. No. 7,778,383 entitled “Effective dual-energy x-ray attenuation measurement” the contents of which is incorporated herein by reference, describes a method of dual energy CT imaging wherein the detector array comprises at least two groups of detector element, each having a different spectral response. At a given source position each of the two groups measures the attenuation in different voxels in the scanned subject. If, for example, alternating detector elements along the rows of detector elements are used to acquire a low energy data set and a high energy data set, the spatial density of samples and resolution for each data set are reduced by a factor of two compared to a conventional scanner. This effect is corrected for in U.S. Pat. No. 7,778,383 patent by switching the focal spot position between two alternating positions.

U.S. Patent application 20100119035 entitled “Computed tomography scanner, in particular for performing a spiral scan, and a method for controlling a computed tomography scanner” the contents of which is incorporated herein by reference, describes a spiral CT scanner wherein radiation filter may be inserted in the X ray beam so it filters a part of the beam and allows simultaneous acquisition with two energy spectra in two different parts of the subject. The '035 application does not disclose how the data so obtain is used to reconstruct dual energy images of same volume.

Multi-slice (or multi-row) CT scanners are known in medical imaging and other applications. In these scanners the detector array comprises a two dimensional array with multiple detector rows disposed in plans parallel to the X-Ray source rotation plane. Multi-slice CT scanners are useful for simultaneous acquisition of multiple slice data or volumetric data. CT scanners with a large number of detectors rows are sometimes referred to as cone beam CT scanners.

Spiral multi-slice CT systems are also known in medical imaging and other applications. In these scanners the scanned subject is translated parallel to the source rotation axis while the source rotates about the subject and attenuation data is acquired.

U.S. Pat. No. 7,551,712 titled “CT detector with non-rectangular cells” to Charles Shaughnessy; discloses a CT detector cell constructed to have diagonally oriented perimeter walls. With such a construction, the resulting CT detector comprised of such detector cells has improved spatial coverage (spatial density).

U.S. Pat. No. 4,352,021; titled “X-Ray transmission scanning system and method and electron beam X-ray scan tube for use therewith”; discloses an X-ray transmission scanning system which uses multiple-anode electrode beam source to provide high speed scanning of body sections. A high speed multiple sections, computed-tomographic x-ray scanner is provided. The scanner utilizes a multiple-anode, scanning electron beam x-ray source to provide high speed scanning of sections of the body. No mechanical motion is involved. Other similar systems, wherein the detector is stationary, are known in the art.

### SUMMARY OF THE EMBODIMENTS

The present invention relates to Computerized Tomography (CT) imaging and more particularly to multiple energy CT imaging.

It is an aspect of embodiments of the invention to provide a CT scanner for multiple energy CT scanning of a subject comprising: an X-Ray source adapted to rotate about the subject; a detector array, having a plurality of detector elements, adapted to acquire attenuation data for X-Rays that have been attenuated by a subject disposed between sais X-Ray source and said detector array, said detector array comprising: at least one region of detector elements having a first spectral response; and at least one region of detector elements having a second spectral response; and a controller, adapted to axially increment the position of said subject respective to said X-Ray source and said detector array such that at least some voxels in the subject that were on lines from said X-Ray source to said detector elements having first spectral response move to lines from said X-Ray source to said detector elements having second spectral response, and wherein the scanner is adapted to generate images based on data associated with at least said first and said second X-Ray energy spectra.

In some embodiments the detector array is an array of detector elements arranged in rows, said rows are substantially parallel to the rotation plane of said source.

In some embodiments alternate rows of detector elements have said first and second spectral response.

In some embodiments the detector elements in said regions of first and second spectral responses have different X-Ray absorption efficiency.

In some embodiments at least one said detector region of a spectral response is shaded from the X-Ray source by partially absorbing X-Ray filters, and wherein at least one detector region of a different spectral response is not shaded from the X-Ray source by said X-Ray filters.

In some embodiments the X-Ray filters are arranged in strips facing the detector elements.

In some embodiments the data is acquired while said subject is at a first position; the subject is axially incremented relative to said X-Ray source and detector array to a second position; and additional data is acquired while the subject is at a said second position.

In some embodiments the data is acquired while said subject is being moved relative to said X-Ray source and detector array.

In some embodiments the detector array comprises two regions of different spectral sensitivities and wherein the CT scanner is adapted to acquire subject attenuation data and reconstruct images associated with two X-Ray energy spectra.

In some embodiments the detector array comprises at least three regions of different spectral sensitivities and wherein the CT scanner is adapted to acquire subject attenuation data and reconstruct images associated with at least three X-Ray energy spectra.

In some embodiments the subject is a human patient.

In some embodiments the subject is luggage.

In some embodiments the attenuation data associated with multiplicity of X-Ray energy spectra are used to determine effective atomic number of material within said subject.

It is another aspect of embodiments of the current invention to provide a CT scanner for multiple energy CT scanning of a subject comprising: an X-Ray source adapted to rotate about the subject; a detector array adapted to acquire attenuation data for X-Rays that have been attenuated by the subject disposed between said X-Ray source and said detector array, said detector array comprising at least one region of a first spectral response and at least one region of a second spectral response.

In some embodiments, the CT scanner further comprises a controller adapted to estimate for a detector region of a first spectral response the attenuation data that would have been measured had this region have a second spectral response, wherein said scanner is adapted to generate images associated with multiplicity of X-Ray energy spectra.

In some embodiments, the CT scanner further comprises a controller capable of axially shift the position of the focal spot respective to said scanned subject and said detector array such that at least some voxels in the subject that were on lines from said X-Ray source to said detector elements having first spectral response move to lines from said X-Ray source to said detector elements having second spectral response. In some embodiments the detector array is an array of detector elements arranged in rows, said rows arranged in planes parallel to rotation plane of said X-Ray source.

In some embodiments the rows of detector elements comprise at least a first section with detector elements of a first spectral response and at least a second section with detector elements of a second spectral response.

In some embodiments the detector elements of a first spectral response are arranged symmetrically to detector elements of a second spectral response respective a plan passing through a focal spot of said X-ray source and the rotation axis.

In some embodiments the scanner is adapted to reconstruct images of multiple energy spectra from attenuation data received during at least 360° rotation of said X-Ray source.

In some embodiments the scanner is adapted to scan said subject by succession of rotational scan each at a fixed subject position.

In some embodiments the scanner is adapted to scan said subject by spiral scan.

In some embodiments the detector area having a first spectral response is different than the detector area having a second spectral response.

In some embodiments the detector area of a first spectral response is larger by a factor of at least four than the detector area of a second spectral response.

In some embodiments the detector area of a first spectral response is larger by a factor of at least ten than the detector area of a second spectral response.

In some embodiments the scanner is adapted to generate images of a first spatial resolution for a first energy spectrum and images of a second spatial resolution for a second energy spectrum.

In some embodiments the detector regions of first and second spectral responses are associated with detector elements of different spectral sensitivities.

In some embodiments the detector regions of first and second spectral responses are associated with detector elements of different efficiency.

In some embodiments at least some of said detector elements belonging to said regions of first and second spectral responses are elements having different dimensions.

In some embodiments the detector regions of first and second spectral responses are associated with different X-Ray beam filtering.

In some embodiments the detector regions of first and second spectral responses are associated with detectors of different spectral sensitivities and different beam filtering.

Unless otherwise defined, all technical and scientific terms used herein have the same meaning as commonly understood by one of ordinary skill in the art to which this invention belongs. Although methods and materials similar or equivalent to those described herein can he used in the practice or testing of the present invention, suitable methods and materials are described below. In case of conflict, the patent specification, including definitions, will control. In addition, the materials, methods, and examples are illustrative only and not intended to he limiting.

Unless marked as background or art, any information disclosed herein may be viewed as being part of the current invention or its embodiments.

### BRIEF DESCRIPTION OF THE DRAWINGS

For a better understanding of the invention and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments of the present invention only, and are presented in the cause of providing what is believed to be the most useful and readily understood description of the principles and conceptual aspects of embodiments of the invention. In this regard, no attempt is made to show structural details in more detail than is necessary for a fundamental understanding of the embodiments; the description taken with the drawings making apparent to those skilled in the art how the several forms of the invention may be embodied in practice. In the accompanying drawings:

FIG. 1A is a front view illustration of prior art multi-slice CT scanner.

FIG. 1B is a side cross-sectional view illustration of prior art multi-slice CT scanner.

FIG. 2A is a side cross-sectional view schematically illustrating a multi-energy CT system according to an exemplary embodiment of the present invention.

FIG. 2B schematically illustrates a top view of a detector array according to an exemplary embodiment of the present invention.

FIG. 2C schematically illustrates a top view of a detector array according to another exemplary embodiment of the current invention, wherein groups of detector elements of different spectral response are arranged in rows of different width in the axial direction.

FIG. 3A(i) schematically illustrates a method for obtaining a multi-energy CT image having increase the sampling density in the axial direction according to an exemplary embodiment of the current invention.

FIG. 3A(ii) schematically illustrates a method for obtaining a multi-energy CT image having increase the sampling density in the axial direction according to another exemplary embodiment of the current invention.

FIG. 3B(i) schematically illustrates a side cross-sectional view of multi-energy CT system performing a method for obtaining a multi-energy CT image having increase the sampling density in the axial direction according to an exemplary embodiment of the current invention.

FIG. 3B(ii) is another schematic illustration of the side cross-sectional view of multi-energy CT system seen in FIG. 3B(i).

FIG. 3B(iii) schematically illustrates a side cross-sectional view of multi-energy CT system performing the method for obtaining a multi-energy CT image having increase the sampling density in the axial direction of FIG. 3A(ii) according to another exemplary embodiments of the current invention.

FIG. 3C schematically illustrates a side cross-sectional view of multi-energy CT system according to another exemplary embodiment of the current invention.

FIG. 4A depict the axial position of detector rows relative to the subject as a function of rotation angle when a prior art system of FIG. 1A and FIG. 1B is used in spiral mode.

FIG. 4B depicts the detector row positions of a multi-energy CT system used in spiral mode according to exemplary embodiments of the present invention.

FIG. 5A is a side cross-sectional view, schematically illustrating a multi-energy CT system according to another exemplary embodiment of the present invention.

FIG. 5B schematically depicts top view of a detector array according to an exemplary embodiment of the current invention.

FIG. 5C schematically depicts a top view of a detector array in accordance with another exemplary embodiment of the invention.

FIG. 5D schematically illustrates a top view of an exemplary detector array useful for dual energy scanning with a system such as seen in FIG. 5A according to another exemplary embodiment of the invention.

FIG. 6A schematically depicts a method wherein the system of FIG. 5A with detector seen in FIG. 5B may be used to generate dual energy images according to an exemplary embodiment of the current invention.

FIG. 6B schematically depicts a method wherein the system of FIG. 5A with detector seen in FIG. 5C may be used to generate dual energy images according to an exemplary embodiment of the current invention.

FIG. 7A schematically depicts a top view of yet another exemplary detector array useful in connections with systems such as system seen in FIG. 5A for acquisition of dual energy data according to an exemplary embodiment of the current invention.

FIG. 7B schematically depicts a method wherein system with detector seen in FIG. 7A may be used to generate dual energy images according to an exemplary embodiment of the current invention.

FIG. 7C schematically depicts a to view of a detector array comprising interleaved detector elements of different spectral response wherein the area covered by both groups is similar according to yet another exemplary embodiment of the current invention.

FIG. 8A is a schematic view of a detector array as known in the art.

FIG. 8B schematically depicts a top view of another exemplary detector for acquisition of dual energy data according to an exemplary embodiment of the current invention.

FIG. 8C schematically depicts a top view of yet another exemplary detector array for acquisition of dual energy data according to an exemplary embodiment of the current invention.

### DESCRIPTION OF THE PREFERRED EMBODIMENTS OF THE INVENTION

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways.

To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like) or multiple pieces of hardware. Similarly, the programs may be stand alone programs, may be incorporated as subroutines in an operating system, may be functions in an installed software package, and the like.

Also, it is to be understood that the phraseology and terminology employed herein is for the purpose of description and should not be regarded as limiting.

In discussion of the various figures described herein below, like numbers refer to like parts. The drawings are generally not to scale.

For clarity, non-essential elements may have been omitted from some of the drawings.

The present invention, in some embodiments thereof, relates to Computerized Tomography (CT) imaging and, more particularly, but not exclusively, to multiple energy CT imaging methods and systems.

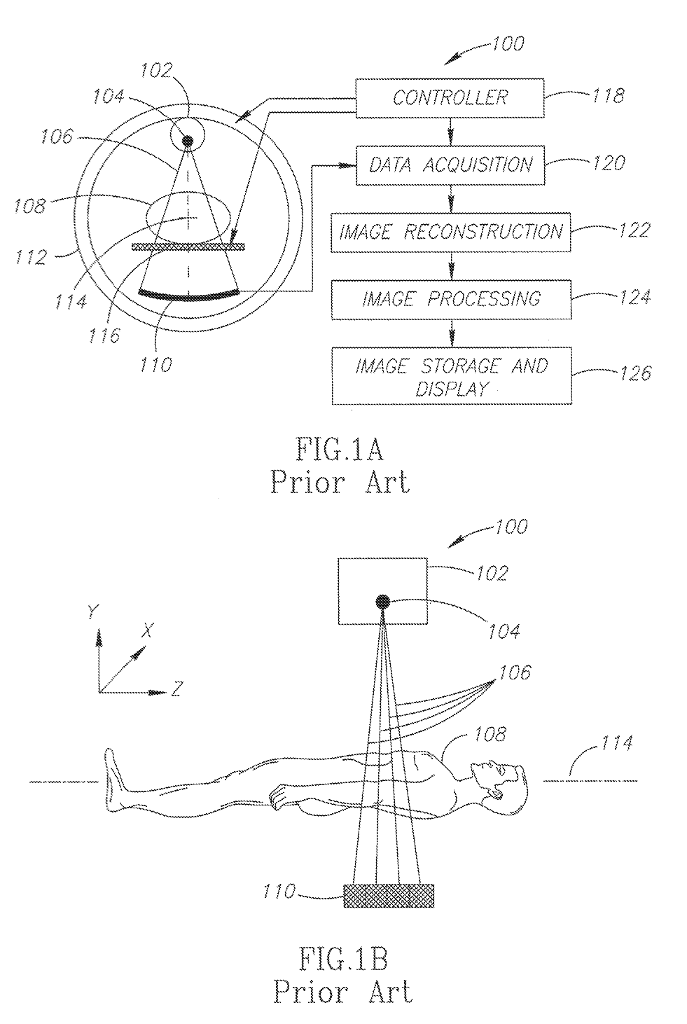

FIG. 1A is a schematic illustration of prior art multi-slice CT scanner 100. X-Ray source 102 comprises a focal spot 104 from which X-Ray beam 106 is emitted. The beam is attenuated by subject 108 and impinges on detector array 110. X-Ray source 102 and detector array 110 are mounted on a rotating frame 112 and made to rotate about rotation axis 114 (along the Z direction) while acquiring attenuation data from multiple view angles. Subject 108, for example a patient in the case of medical imaging or suspected item such as luggage in security imaging, is supported by support 116. Support 116 may be for example a motorized patient table in the case of medical imaging or a conveyor in security imaging, or the likes. Patient position, source rotations and other functions of system 100 are controlled by control unit 118. Attenuation data acquired by data acquisition sub-system 120 is reconstructed to three dimensional (3D) images by image reconstruction sub-system 122, wherein the images are optionally processed further by image processing sub-system 124 and optionally stored and/or displayed by image storage and display sub-system 126. The gantry frame, beam collimation, and various other parts of the scanner which are not material for understanding of the invention are not shown in FIG. 1A. In some CT scanners, detector array 110 has a curved arc shaped front surface as shown in FIG. 1A. In some CT scanners detector 110 is flat, curved, or has other front surface shape.

FIG. 1B is a side cross-sectional view illustration of prior art multi-slice CT scanner 100.

The detector array 110 is a two dimensional (2D) detector array extending in the {X, Z} directions (here Z is parallel to the rotation axis 114 of rotating frame 112) where one column of detector elements out of the plurality of parallel columns is shown in FIG. 1B.

System 100 is a multi-slice scanner with four slices in the particular example shown. During irradiation and data acquisition four sets of attenuation data are acquired by the four rows of detectors. Scanners of different number of detector rows are known in the industry and available commercially from multiple vendors. E.g. CT scanner model “Equillion One” from Toshiba Medical Systems has a detector with 320 rows of detector elements. The attenuation data from multiple view angles are reconstructed to multiple slice or volumetric images using algorithms known in the art. Common algorithms for image reconstruction of fan beam or cone beam CT scanners include preprocessing the raw detector data, convolution of the data along rows of detector elements with filter function and back-projection of the filtered data to images. However other algorithms may be used as well. Filter Back-Projection (FBP) algorithms typically require data from at least 180° view angles. Preferably, the entire scanned object is contained in the scan field as defined by the X-ray beam and detector coverage so view data are not truncated.

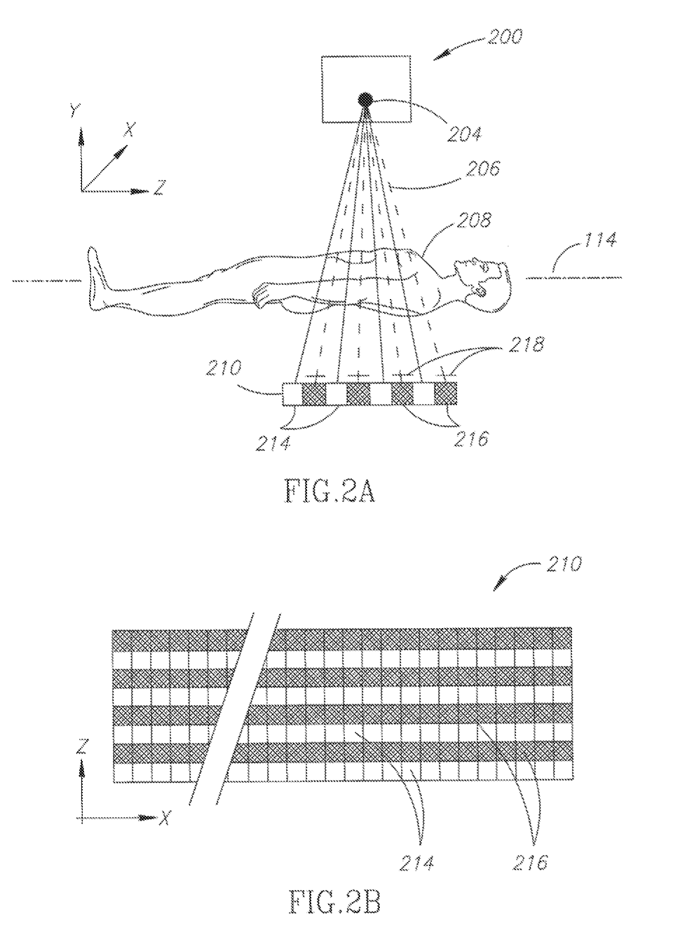

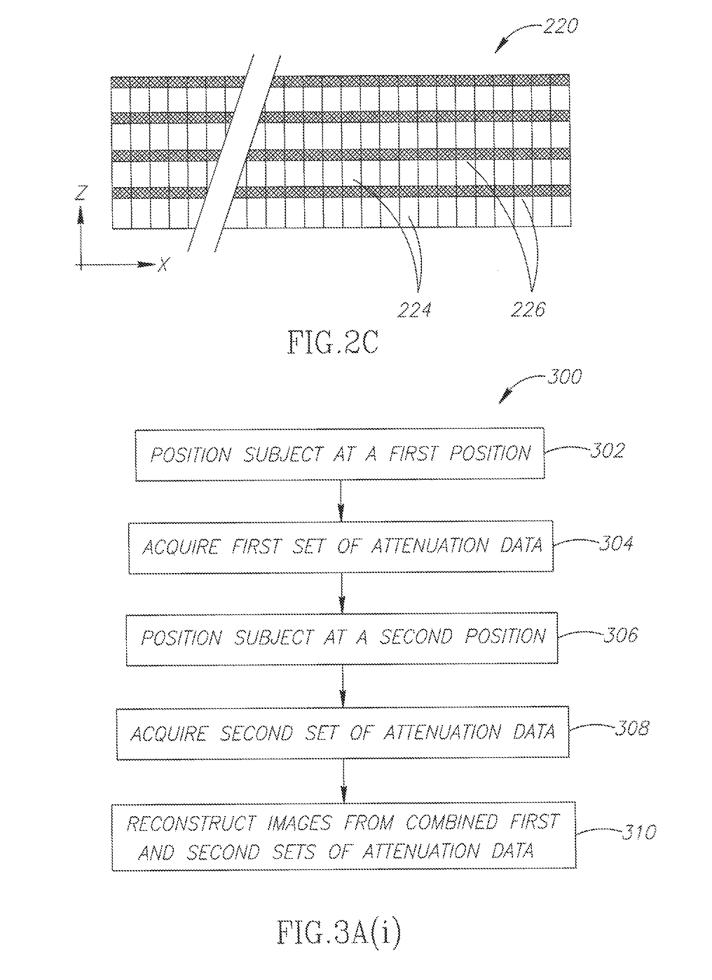

FIG. 2A is a side cross-sectional view schematically illustrating a multi-energy CT system 200 according to an exemplary embodiment of the present invention.

Focal spot 204 emits X-Ray beam 206. Unlike detector 110 of FIG. 1B which comprises substantially identical detector elements across the detector array, detector array 210 comprises two types of detector elements 214 and detector elements 216 which are different in their spectral response.

FIG. 2B schematically illustrates a top view of detector array 210 according to an exemplary embodiment of the present invention.

Groups of detector elements 214 and detector elements 216 are arranged in this example in total of eight interleaved detector rows, four rows of each type. In FIG. 2B, groups of detector elements 214 and detector elements 216 are arranged in rows of same width in the Z direction.

FIG. 2C schematically illustrates a top view of detector array 220 according to another exemplary embodiment of the current invention, wherein groups of detector elements 224 and detector elements 226 of different spectral response are arranged in rows of different width in the Z direction.

System 200 also comprises sub assemblies such as subject support 116, controller 118, data acquisition unit 120, reconstruction unit 122, image processing unit 124 and image storage and display unit of FIG. 1A and other sub-assemblies common to CT scanners which are not shown for drawing clarity.

Detector elements 214 and detector elements 216 are different in their spectral response. By different spectral response is meant, for example, that if the detectors are exposed to X ray beam of a wide spectrum, one group of detector elements is adapted to response more efficiently to a lower energy part of the spectrum compared to the other group. In exemplary embodiments detector array 210 comprises array of scintillator crystals adapted to absorb X-Rays and convert the absorbed X-Ray energy to Infra-Red, visible or UV light. The scintillator crystals are optically coupled to photodiode array adapted to convert the light to electrical signals. In exemplary embodiments detector elements 214 comprise relatively low absorbing scintillator material such as ZnSe, providing good sensitivity to low energy X-Ray photons and poor sensitivity to high energy X-Ray photons. In exemplary embodiments detector elements 216 comprise relatively high absorbing scintillator material such as CdWO4 or GOS, providing high sensitivity to all X-Ray photons. With this arrangement, for a given spectrum of X-rays emitted by the source, the average energy of photons observed by detector elements 214 is lower than the average energy observed by detector elements 216.

In another exemplary embodiments the difference between the spectral responses of detector elements 214 and detector elements 216 is further enhanced by placing partially-absorbing radiation filters 218 (seen in FIG. 2A) between the X-Ray source and one type of detector elements while not placing filters, or placing different filters in front of the other type of detector elements. In some embodiments filters 218 which reduce the low energy component of the X-Ray spectrum are placed in front of detector elements intended to measure higher energy X-Rays while same filters are not placed in front of the detector elements intended to measure lower energy X-Rays.

In some embodiments the filters are attached to the detector elements, in other embodiments the filters are positioned at a distance from the detector elements such that the filter elements shadow the detector elements from the X-Ray source.

In the detector layouts such as shown in FIG. 2B or FIG. 2C, filters 218 may be arranged as strips facing detector rows. In some embodiments the detector elements 214 and detector elements 216 have same structure and the different in spectral response is achieved by placing filters 218 in front of one group of detector elements only.

In an exemplary embodiment useful for medical imaging, the X-Ray source is an X-Ray tube operating at tube voltage of 140 KV without added filtering except for beam filtering by the components of the tube. Source to axis distance is 570 mm and source to detector distance is 1040 mm. Optionally there are 8 detector rows, where four rows comprise ZnSe crystals of 1.2 mm height and four interleaved rows comprising CdWO₄ crystals of same height. The pitch between rows centers in this example is 1.37 mm, corresponding to pitch between rays of 0.75 mm at the axis of rotation. Radiation filter elements made of 1 mm thick titanium are optionally placed in front of the CdWO₄ rows of detector elements. Persons with common knowledge of the art will appreciate this quantitative description is given by a way of example and other dimensions, X-Ray source types and mode of operation, number of detector rows, type and composition of detector elements, design of optional filters and other design parameters may be used in the framework of the invention.

A single rotation scan using system 200 of FIG. 2A would generate for each type of detector elements 214 or detector elements 216, data of certain sampling density in the axial direction Z wherein said sampling density depends on the center-to-center distance for detector rows of one type of detector elements. For various applications it may be desired to increase the sampling density in the axial direction.

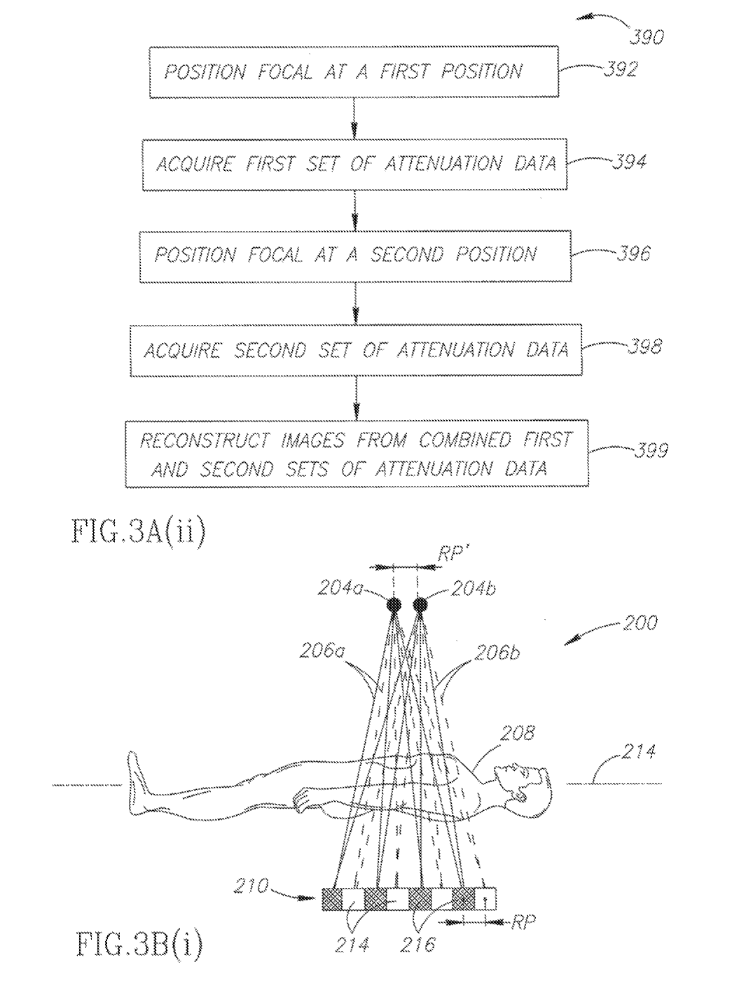

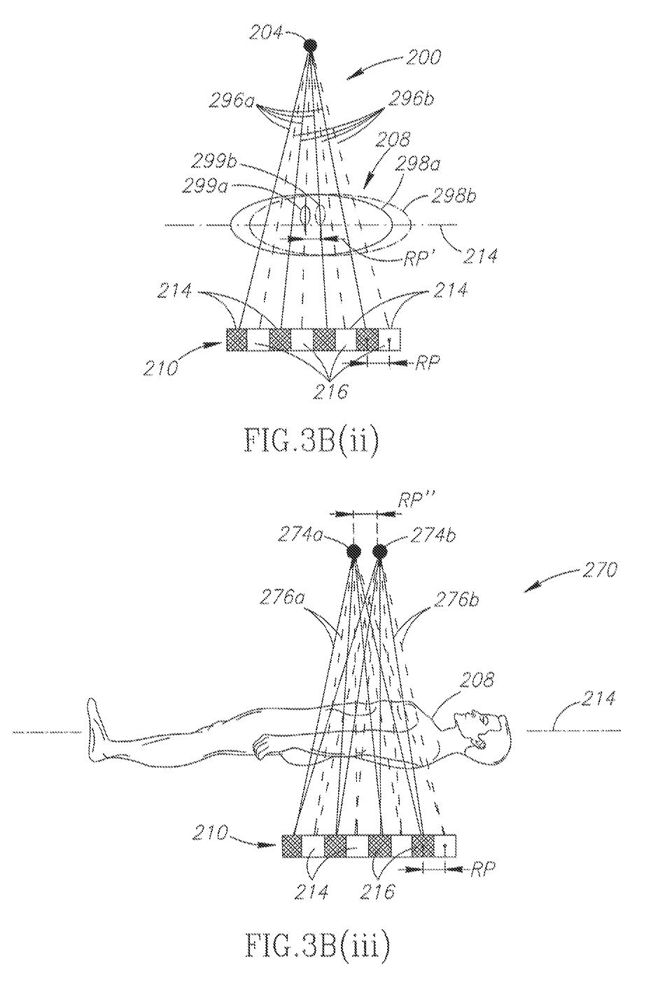

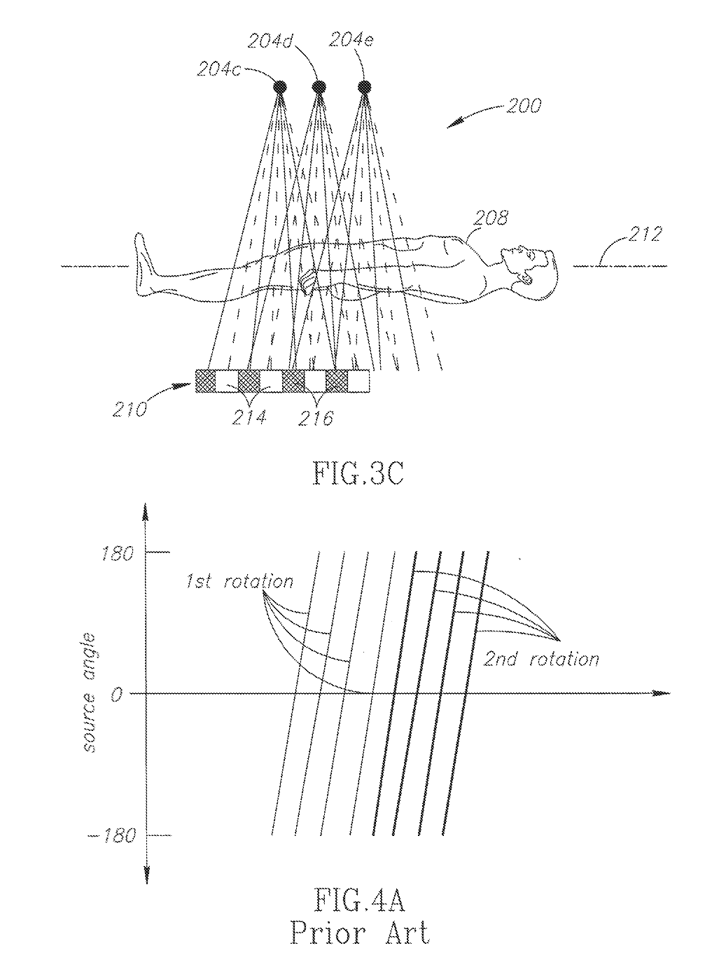

FIG. 3A schematically illustrates a method 300 for obtaining a multi-energy CT image having increase the sampling density in the axial direction according to an exemplary embodiment of the current invention.

FIG. 3B(i) schematically illustrates a side cross-sectional view of multi-energy CT system 200 performing the method 300 for obtaining a multi-energy CT image having increase the sampling density in the axial direction according to an exemplary embodiment of the current invention.

According to exemplary embodiments of the invention illustrated in FIG. 3A(i); 3B(i) and FIG. 3B(ii) , system 200 is used in the following “step and shoot” scheme in order to increase the sampling density in the axial Z direction for each detector elements type:

In first subject positioning step 302 of FIG. 3A(i), subject 208 is positioned by controller 118 at a first position relative to the scanner frame, noted by the focal spot position 204a relative to subject 208 in FIG. 3B(i). Detector array 210 is shown opposing focal spot 204a.

In first data acquisition step 304 attenuation data of X-ray beam 206a is acquired at the first subject position. During the data acquisition step 304, rotating frame 112 rotates for example by a full rotation or a rotation of 180 degrees plus the angular spread of X-Ray beam 106 seen in FIG. 1A in order to acquire a complete data set for reconstruction using FBP algorithm.

In second subject positioning step 306 the subject is moved to a second position relative to the scanner frame in the axial direction, noted by the focal spot position 204b relative to subject 208 in FIG. 3B(i). For example, subject 208 may be moved in the axial direction Z by an increment RP′, where RP′ (the effective row Pitch) is the axial distance between centers of adjacent rows RP (rows of different types) as projected onto the rotation axis 214 as seen in FIG. 3B(i). Thus, at least some voxels in the subject 208 that were on X-Ray path from focal point 204a to detector elements 214 are now on X-Ray path from focal point 204b to detector elements 216 and vice versa. RP′ is measured as the pitch between detector rows as projected on the rotation axis.

For drawing clarity, relative motion of subject 208 to focal X-ray source and the detector is demonstrated in the figure by translating the focal point 204. For drawing clarity the detector is not shown shifted.

In second data acquisition step 308 attenuation data of X-ray beam 206b are acquired at the second subject position similarly to the data acquisition in the first data acquisition step 304.

Optionally, for imaging a long axial range of subject 208, the axially shift of the subject 208 may be repeated by distance RP′ and data acquisition steps repeated.

In image reconstruction step 310 the combined data from first and second (and optionally from more) subject positions are optionally reconstructed to separate 3D images for each of the spectral responses and processed using algorithms known in the art.

In exemplary embodiments with multiple rows of detectors such algorithms may include:
i. preprocessing raw data to corrected and calibrated logarithmic data;
ii. convolution of the data for each view angle with filter function, said convolution may be performed along rows of detector elements; and
iii. back-projection of filtered data, said back-projection of first and second sets of data are according to first and second positions of the subject. Other algorithms and adaptations thereof may be used as well.

The dual energy images so achieved may be displayed, archived and processed further to analyze the material composition of the subject along with the CT numbers. As known in the industry, dual energy material composition analysis may be done also from pre-processed data before image reconstruction.

FIG. 3B(ii) is another schematic illustration of the side cross-sectional view of multi-energy CT system seen in FIG. 3B(i).

In this figure, focal point 204 and detector array 210 are seen stationary, as are the X-Ray paths 296a from focal point 204 to detector elements 214 of the first type and -Ray paths 296b from focal point 204 to detector elements 26 of second type.

Subject 208 is seen in its first position 298a as in positioning step 302 and after it was moved along axis 214 by a distance RP′ to its second position 298b as in positioning step 306. Consequently, a voxel within the subject has been moved from its first position 299a on path 296b to its second position 299b on path 296a.

FIGS. 3A(ii) and 3B(iii) depict another method and system for obtaining a multi-energy CT image.

FIG. 3A(ii) schematically illustrates a method for obtaining a multi-energy CT image having increase the sampling density in the axial direction according to another exemplary embodiment of the current invention.

FIG. 3B(iii) schematically illustrates a side cross-sectional view of multi-energy CT system performing the method for obtaining a multi-energy CT image having increase the sampling density in the axial direction of FIG. 3A(ii) according to another exemplary embodiments of the current invention.

According to another exemplary embodiments of the invention illustrated in FIG. 3A(ii) and 3B(iii), system 270 is used system performing the method for obtaining a multi-energy CT image having increase the sampling density in the axial direction.

System 270 comprises a in the following “dual focal spots” X-Ray source, wherein focal spots 274a and 274b are shifted in the axial z direction. Such dual focal spot X-Ray source may be for example a sources (as known in the art, e.g. with the x-ray tube “Straton” by Siemens GMBH) scheme in order to increase the sampling density in the axial Z direction for each detector elements type. Alternatively, a mechanical motion apparatus may be used to shift the X-Ray tube.

In first focal spot positioning step 392 of FIG. 3A(ii), subject 208 is positioned by controller 118 at the desired position relative to the scanner frame, noted by the focal spot position 274a relative to subject 208 in FIG. 3B(iii). Detector array 210 is shown opposing focal spot 2704a.

In first data acquisition step 394 attenuation data of X-ray beam 276a is acquired at the first focal spot position. During the data acquisition step 3394, rotating frame 112 rotates for example by a full rotation or a rotation of 180 degrees plus the angular spread of X-Ray beam 106 seen in FIG. 1A in order to acquire a complete data set for reconstruction using FBP algorithm.

In second focal spot positioning step 396 the focal spot is moved to a second position relative to the scanner frame and the patient in the axial direction, noted by the focal spot position 274b relative to subject 208 in FIG. 3B(iiii). For example, focal spot 274 may he moved in the axial direction Z by an increment RP″, where, in this embodiment, RP″ is the axial distance between centers of adjacent rows RP (rows of different types) as projected onto the focal spot radius (the effective row Pitch at the rotation axis 214 as seen in FIG. 3B(iii) multiply by the ratio between the focal spot to axis of rotation distance to the detectors to the axis of rotation distance). Thus, at least some voxels in the subject 208 that were on X-Ray paths 276a from focal point 274a to detector elements 214 are now on X-Ray paths 274b from focal point 274b to detector elements 216 and vice versa.

In second data acquisition step 398 attenuation data of X-ray beam 276b are acquired at the second focal spot position 274b similarly to the data acquisition in the first data acquisition step 394.

Optionally, the data acquisition steps 394 could be executed for one view or for few views, then switched to step 936 and vice versa, until completion acquire a minimum complete data set for reconstruction.

Optionally, for imaging a long axial range of subject 208, an axially shift of the subject 208 may be repeated by distance 2*RP″ and data acquisition steps repeated.

In image reconstruction step 399 the combined data from first and second (and optionally from more) subject positions are optionally reconstructed to separate 3D images for each of the spectral responses and processed using algorithms known in the art.

FIG. 3C schematically illustrates a side cross-sectional view of multi-energy CT system according to another exemplary embodiment of the current invention.

FIG. 3C illustrates another exemplary scanning scheme using system 200, where it is desired to scan a wide volume of the subject. First patient position relative to the source is shown as focal point 204c opposing detector 210. A second patient position is achieved by incrementing the patient by 3RP′ relative to the scanner frame. A third patient position is achieved by incrementing the patient by additional 3RP′ relative to the scanner frame, and so forth till the desired subject width is covered. Different inter-scan patient increment may be used depending on the number of detector rows, desired coverage and other parameters.

System 200 is shown as having a detector array 210 with eight rows of detector elements with uniform pitch between detector rows and two types of detector elements arranged in alternating rows. Other detector layout schemes and other schemes of subject increment relative to scanner frame can also be used such that interleaved attenuation data of different spectral response is generated. In particular, three types or more of detector elements with different spectral sensitivities may be used to generate multiple energies scan data. Also, detector elements of same spectra response may optionally be arranged in groups different that alternating rows. For example, there may be alternating groups of detector elements, each group comprising multiple rows of detector elements of same spectral response.

Spiral (or helical) CT scanning is well known in the art. In spiral scanning the subject is moved parallel to the rotation axis while the X-Ray source, or both the X-Ray source and detector array, rotate about the subject and attenuation data is acquired.

FIG. 4A depict the Z position of detector rows relative to the subject as a function of rotation angle when a prior art system 100 of FIG. 1A and FIG. 1B is used in spiral mode.

For clarity, detector row positions at a first rotation are marked in FIG. 4A as thin lines and detector row positions at a second rotation are marked as thick lines. However, these lines represent any two of multiple continuous rotations. The subject speed relative to the scanner frame is selected in this example such that the subject increment per rotation in FIG. 4A is 4RP′, wherein RP′ as defined herein above. This spiral speed is known in the art as pitch=1 for the exemplary 4 slice CT. The spiral attenuation data may be reconstructed to slice or volume images by algorithms known in the art.

FIG. 4B depicts the detector row positions of exemplary system 200 used in spiral mode according to exemplary embodiments of the present invention.

Alternating solid and dashed lines present the positions of detector element rows belonging to two groups, e.g. 214 and 216 respectively as described herein above. Thin lines present detector row positions in a first rotation and thick lines present detector row positions in a second continuous rotation. For clarity of the figure, the lines of the second rotation are shown slightly off the lines of the first rotation, although for exemplary patient increment per rotation of 3RP′ they are overlapping.

In exemplary embodiments attenuation data are processed separately for groups of detector elements 214 and detector elements 216. As seen in FIG. 4B, for certain subject speeds, for example 3RP′ per rotation for exemplary system 200, there is at least a section of the scanned subject where spiral data is generated for each of detector types 214 and 216 with same sampling density in the axial direction as may be obtained for example in prior art system 100 when used according to FIG. 4A. System 200 used in spiral mode as describes herein provides simultaneously two data sets for two energy spectra while maintaining the sampling density in the Z direction of prior art systems of comparable row to row pitch RP.

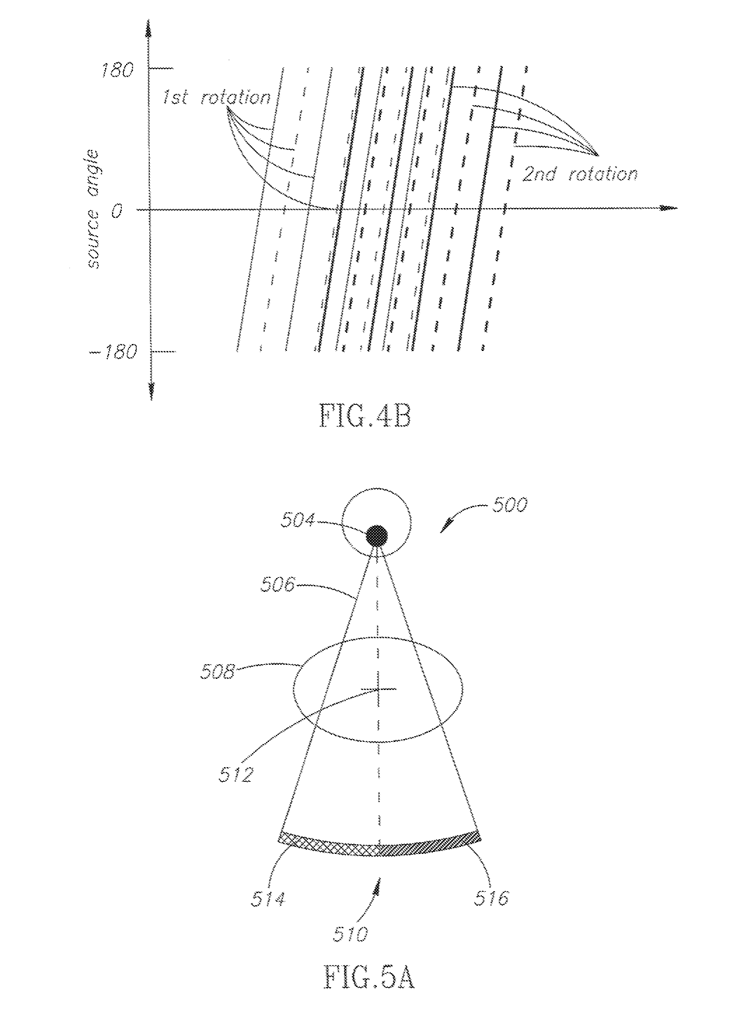

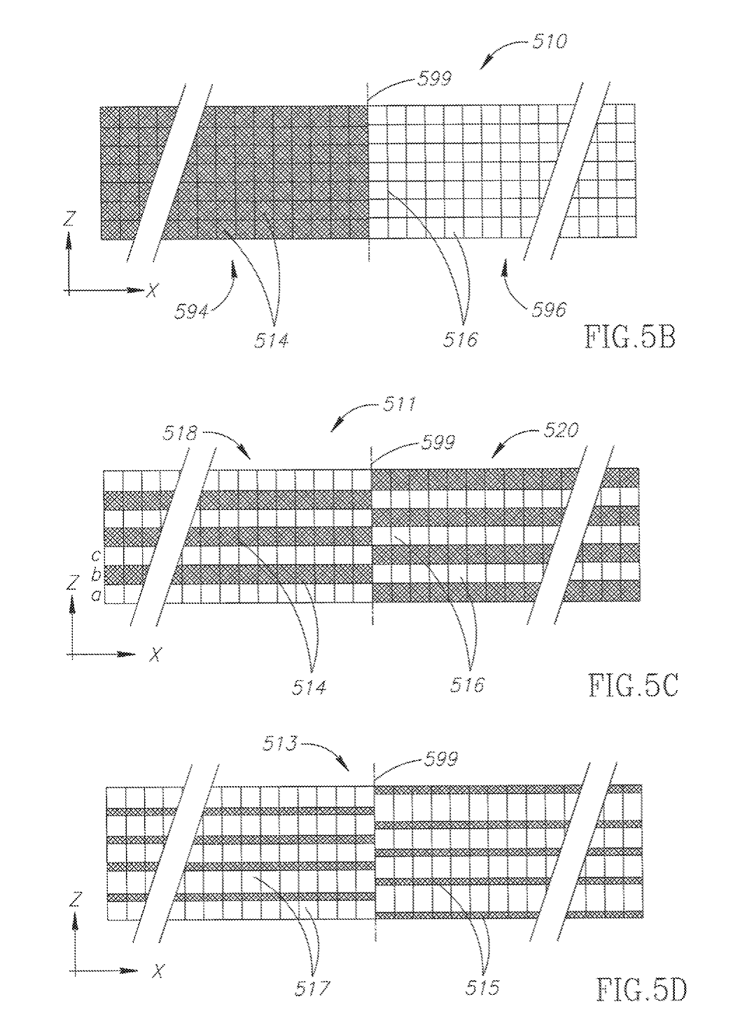

FIG. 5A is a side cross-sectional view, schematically illustrating a multi-energy CT system 500 according to another exemplary embodiment of the present invention.

System 500 comprises an X-ray source having a focal point 504 which emits X-ray beam 506. X-rays 506 are attenuated by subject 508 are detected by detector array 510. X-Ray source with focal point 504 and detector 510 are adapted to rotate about rotation axis 512 while attenuation data is collected from multiple view angles. Various other parts of the systems are not shown for clarity. In exemplary embodiment detector array 510 comprises two groups of detector elements 514 and 516, each with a different spectral response.

FIG. 5B schematically depicts top view of exemplary detector array 510 according to an exemplary embodiment of the current invention.

In this embodiment detector array 510 comprises a first region 594 with detector elements type 514 and a second region 596 with detector elements type 516. The border 599 between the first and second regions 594 and 596 is along the crossing of the detector top surface with imaginary plan defined by the focal spot 505 and the axis of rotation 512. It should be noted that if array 510 is not split to the two regions exactly in the center, one of the regions may not acquire enough data to reconstruct full image as the data acquired by it is missing information on voxels in the vicinity of the rotation axis 512.

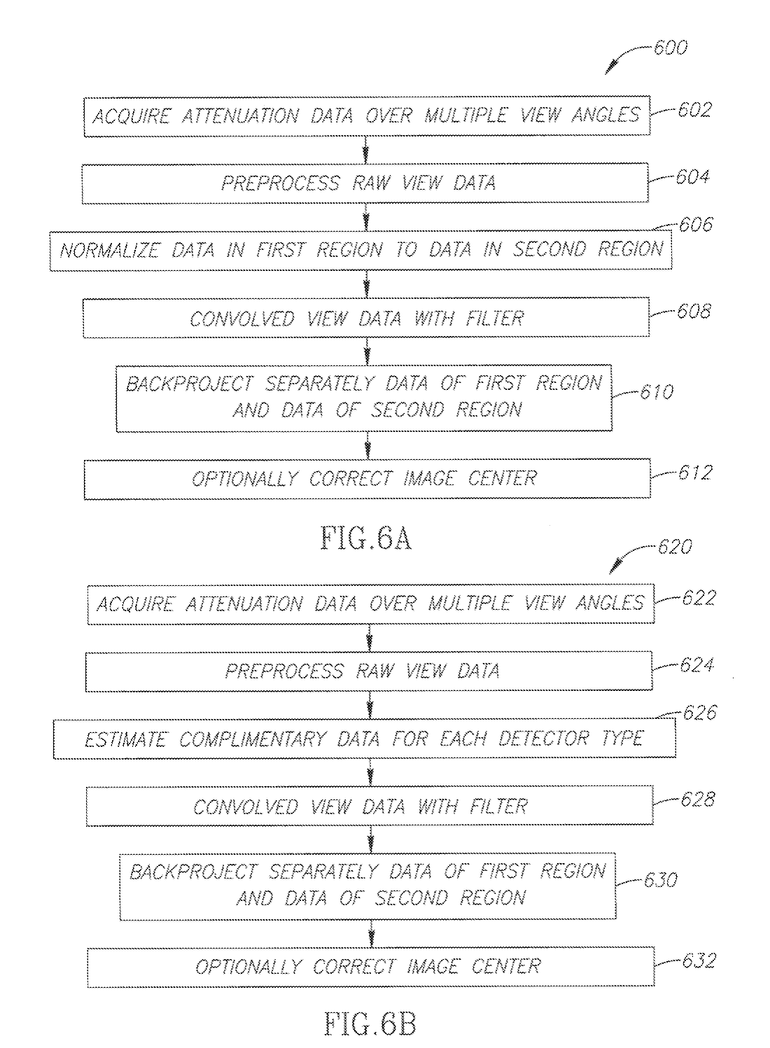

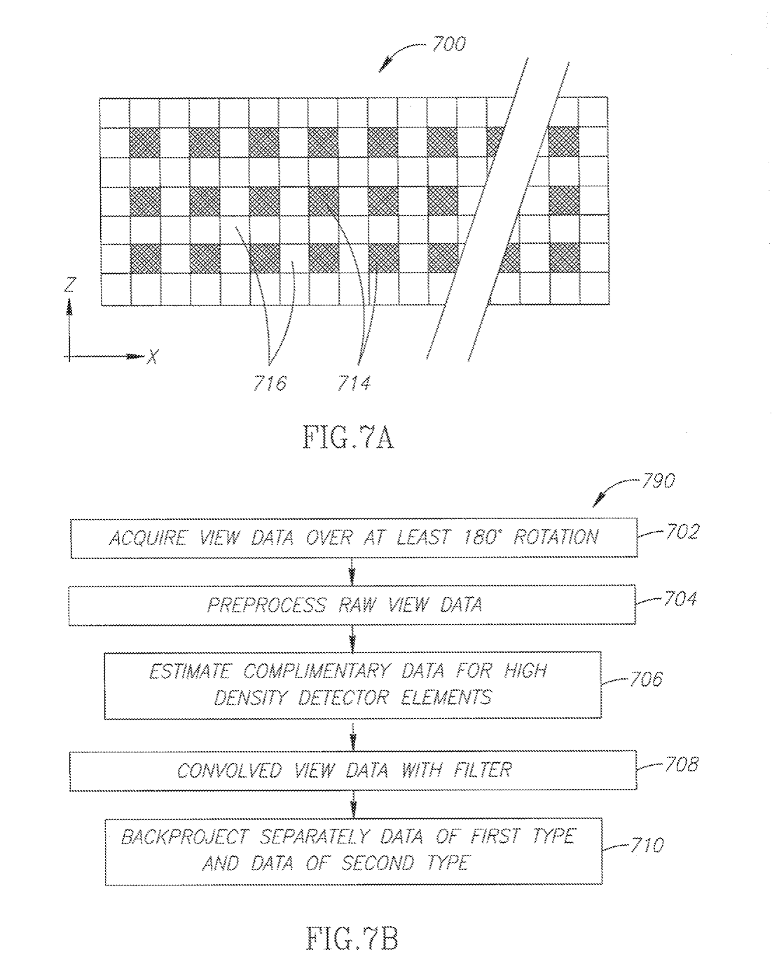

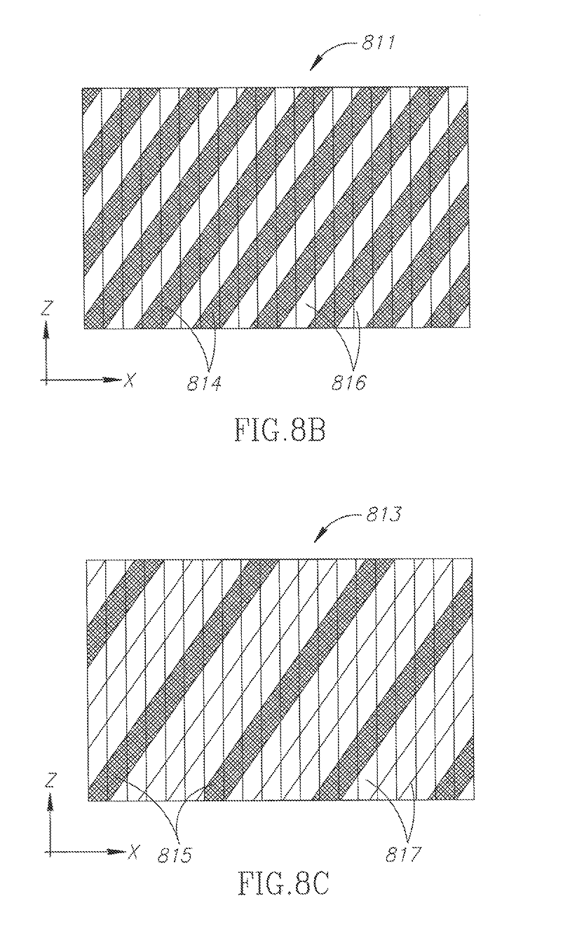

FIG. 6A schematically depicts a method 600 wherein system 500 of FIG. 5A with detector 510 may be used to generate dual energy images according to an exemplary embodiment of the current invention.

In data acquisition step 602 attenuation data are acquired for multiple view angles covering 360° or more around the subject.

In pre-processing step 604 the view data are calibrated, corrected and converted to logarithmic data as commonly done in the art.

In normalization step 606 the data in one detector region is normalized to the data in the other detector region (e.g. data of elements 514 is normalized to data of elements 516, or vice versa). This step is used as detector groups 514 and 516 may have different signal level for a given cross section of the attenuating subject 508.

In convolving step 608 the normalized view data are convolved with a filter function along detector rows as known in the art. In this step, the normalized view data of one group of detector elements (e.g. 514) are used to estimate the data that would have been obtained by detectors of the other group (e.g. 516) at same positions in the array, and vice versa, for the purpose of convolving the filter with complete non-truncated views.

In back-projection step 610 the data of the first and second regions are back-projected separately to form first and second sets of images of the scanned subject, corresponding to different energy spectra. As known in the art, 180° view data are sufficient for image reconstruction. The geometry and procedure described herein provide 180° of data across the entire scan volume for each of the detector groups.

The procedure described herein may yield images with artifact in the center of the image, corresponding to the center of rotation. In optional step 612, image artifacts may be corrected by interpolating the image around the center to the center.

FIG. 5C schematically depicts a top view of detector array 511 in another exemplary embodiment of the invention.

In array 511, detector elements 514 and detector elements 516 are arranged in alternating rows wherein each row comprises elements of a first type in a first part of the row and elements of a second type in a second part of the row. Detector array 511 comprises a first region 518 of the detector array and a second region 520 of the detector array wherein the rows with elements of certain group are shifted between the regions. The border 599 between the regions 518 and 520 in array 511 is preferably along the crossing of the detector array stop surface with imaginary plan defined by the focal 504 spot and the axis of rotation 512.

FIG. 6B schematically depicts a method 620 wherein system 500 of FIG. 5A with detector 511 may be used to generate dual energy images according to an exemplary embodiment of the current invention.

Steps 622 and 624 are similar to steps 602 and 604 in FIG. 6A, respectively, except that some embodiments may use data of less than 360°.

In estimation step 626, for each detector row, complimentary data is estimated for each detector type. For example, for the row marked as row b in FIG. 5C, directly measured data is available for detector elements type 514 for a first part of the row (left hand side of FIG. 5C). Data for detector elements type 514 is estimated for the second part of the row (right hand side of FIG. 5C), where detector elements type 514 are not available, by interpolating between type 514 data of the rows marked a and c. Nearest neighbor or higher order interpolation may be used, employing also further rows. At edge rows, the complimentary data is extrapolated. This process is repeated for all rows for both types of detector elements. The result of this stage are two sets of data for the entire array, corresponding to two energy spectra, wherein in each set some data is directly measured data and some data is estimated data.

In convolution step 628 the normalized view data are convolved with a filter function along detector rows as known in the art. In this step, the estimated data of each group of detector elements (e.g. 514) are used to complete the view data, for the purpose of convolving the filter with complete non-truncated views.

In back-projection step 630 the data of the first and second data sets are back-projected separately to form first and second sets of images of the scanned subject corresponding to different energy spectra. In some embodiments each entire set of data comprising directly measured and estimated data, is back-projected separately to form images corresponding to a first and second energy spectra. Such embodiments have effective sampling density and spatial resolution in the axial direction which are improved relative e.g. to system 200 described herein above when used for a single acquisition. In other embodiments wherein data is acquired for 360° or more, only directly measured data is used in back-projection wherein for each volume element in the scanned volume there is data available for 180° for each detector type. Such embodiments provide dual energy images of same axial sampling density as prior art systems of similar construction.

The procedure described herein may yield images with artifact in the center of the image, corresponding to the center of rotation. In optional image correction step 632, image artifacts in the center of the image may be corrected by interpolating the image around the center into the center.

FIG. 5D schematically illustrates a top view of exemplary detector array 513 useful for dual energy scanning with system such as 500 according to another exemplary embodiment of the invention.

Detector array 513 comprises two groups of detector elements 515 and 517 with first and second spectral responses. Array 513 has a similar structure to array 511 of FIG. 5C and is used in a similar manner. However, elements 515 and 517 have a different width in the axial direction. This may be useful, for example, to obtain similar output from elements 515 and 517 in a case where one type of detector elements (e.g. 515) is more responsive to the radiation than the other type (e.g. 517).

Persons familiar with the art will appreciate that other algorithms and derivatives thereof may he used to reconstruct dual energy images for system 500 with detector 510, 511, 513 or other detector designs and are covered by the invention. In particular, the procedures 300, 600 and 620 above may he modified to acquire and reconstruct dual energy images in spiral mode. Certain divisions of the detector array were described. In other exemplary embodiments the detector array may he divided to areas of elements of different spectral response in a different way than shown in previous figures.

It should he noted that reconstruction algorithms other than FBP may be used. For example, iterative or algebraic reconstruction algorithm may be used. Additionally, datasets derived from information acquired by the two types of detector elements may be created and reconstructed. For example (but not limited to), the ratio or the difference between the datasets indicative of signals detected in the two types of detector elements may be created and reconstructed.

It should be noted that optionally, only one CT image may be reconstructed from one or from both datasets indicative of signals detected in the two types of detector elements. Information indicative of the differences between the two datasets may be in form of markings, pointer or text giving information derived from the differences. These may be pointing to specific areas in the one reconstructed CT image, or referring to the entire image or slice or section of the image. For example, material composition (for example, high/low atomic number, suspected substance and the likes) may be derived from comparing the two datasets and displayed to the user.

FIG. 7A schematically depicts a top view of yet another exemplary detector array 700 useful in connections with systems such as system 500 for acquisition of dual energy data according to an exemplary embodiment of the current invention.

Detector array 700 comprises interleaved detector elements 714 and detector elements 716 with different spectral responses wherein the area covered by elements 716 is larger than the area covered by detector elements 714. In the exemplary embodiment shown all detector elements 714 and detector elements 716 have same shape and area and each detector element 714 is surrounded by detector elements 716 so there are approximately three times as many 716 elements compared to 714 elements (disregarding the edges of the array). It should be noted that other embodiments may have a different arrangement and different ratios within the scope of the current invention. For example, there may be a ratio of 10 or 20 or 100 or other value between the area covered by one type of detector elements and the area covered by another type of detector elements. Detector groups of elements 714 and elements 716 are termed here as low density and high density detector elements, respectively, referring to the amount of elements in the array.

FIG. 7B schematically depicts a method 790 wherein system with detector 700 may be used to generate dual energy images according to an exemplary embodiment of the current invention.

Steps 702 and 704 are similar to steps 622 and 624 in FIG. 6B.

In estimation step 706, estimate is made for data of high density detector elements type 716, had they been placed the positions of low density detector elements 714. This is done by nearest neighbors or higher order interpolation. The result of this step is a complete high density data set for detector elements 716.

In convolving step 708 the high density data set achieved in estimation step 706 is convolved with a filter function. The low density data set of elements 714 is convolved separately with same or different filter function.

In back-projection step 710 each set of filter convolved data is back-projected separately. The results are high resolution images of a first energy spectrum and lower resolution images of a second energy spectrum.

The high resolution image may be used for visualization and identification of features in the scanned subject. Both set of images may be used together to analyze material composition of features in the scanned subject. Alternatively, material composition may be determined based on pre-processed data. This may be useful in particular if the density of the low density detector elements is too low to reconstruct acceptable quality images.

FIG. 7C schematically depicts a to view of detector array 720 comprising interleaved detector elements 722 and detector elements 724 of different spectral response wherein the area covered by both groups is similar according to yet another exemplary embodiment of the current invention.

In the checkerboard arrangement of array 720, two complete sets of attenuation data may be achieved for each detector type by estimating each missing data point by interpolation from nearest neighbors or by higher order interpolation.

It should be noted that reconstruction algorithms other than FBP may be used. For example, iterative or algebraic reconstruction algorithm may be used. Additionally, datasets derived from information acquired by the two types of detector elements may be created and reconstructed. For example (but not limited to), the ratio or the difference between the datasets indicative of signals detected in the two types of detector elements may be created and reconstructed.

It also should be noted that some algebraic reconstruction algorithm, for example FBP are sensitive to the completeness of the dataset, and may exhibit artifacts if are used on dataset where some spatial sampling is incomplete (for example, but not limited to data acquired by detectors elements 713 of FIG. 7A, or data acquired without the incremental subject translation depicted in FIG. 3B(ii)). In these cases, it may be preferred to apply data estimation (for example, as depicted in FIG. 6B). In contrast, iterative algorithms are more robust and may tolerate some missing data or incomplete dataset without noticeable, or with minor artifacts. Thus, when iterative reconstruction algorithms are used, the step of data estimation may optionally be avoided.

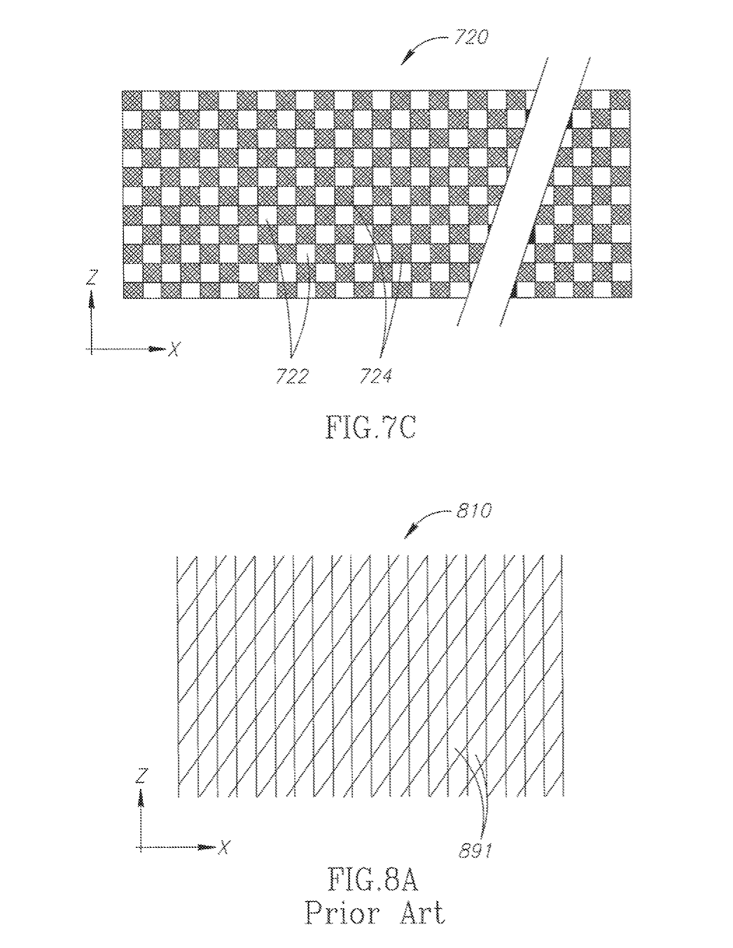

FIG. 8A is a schematic view of a detector array, as described in U.S. Pat. No. 7,551,712 entitled “CT Detector with Non-Rectangular Cells” the contents of which is incorporated herein by reference. U.S. Pat. No. 7,551,712 describes a method to achieve a better special resolution by having a detector array 810 having detector elements 891 with diagonally oriented perimeter walls between the detectors elements.

FIG. 8B schematically depicts a top view of a section of another exemplary detector array 811 useful in connections with systems such as system 500 for acquisition of dual energy data according to an exemplary embodiment of the current invention.

Detector array 811 comprises interleaved diagonally oriented detector elements 814 and detector elements 816 with different spectral responses. Detector array 811 combines the advantage discloses in U.S. Pat. No. 7,551,712 with dual-energy capabilities according to the current invention. The arrangement of FIG. 8B allows for arranging the detector elements in each type in contiguous rows (slanted in respect to the CT axis X and Z). For example, the difference in spectral response of detector elements 814 and 816 may be due to a strip of partially X-Ray absorbing material placed between detector elements 814 and the scanned subject.

FIG. 8C schematically depicts a top view of a section of yet another exemplary detector array 813 useful in connections with systems such as system 500 for acquisition of dual energy data according to an exemplary embodiment of the current invention.

Detector array 813 comprises interleaved diagonally oriented detector elements 815 and detector elements 817 with different spectral responses, similarly to the embodiment depicted in FIG. 8B. However, in detector array 813, the area covered by elements 817 is larger than the area covered by detector elements 815. In the exemplary embodiment shown all detector elements 815 and detector elements 817 have same shape and area but there are approximately three times as many 817 elements compared to 815 elements. It should be noted that these design parameters are for demonstration only and the ratio of element numbers of the two element types, and the ratio between the areas covered by the two element types may vary within the scope of thee current invention.

The invention is described in reference to embodiments with detector arrays divided to detector elements. It shall be appreciated the invention can be used also with other types of detectors wherein the active area of the detector is divided to regions of different spectral sensitivity. The detector array may comprise any number of rows of discrete elements or any number of regions. Certain values of inter-scan or spiral subject increment respective the scanner frame are given by way of examples. However, other values of increments may be used. Any suitable reconstruction algorithm known in the art may he used to reconstruct images out of attenuation data, optionally with adjustment for the structure of the detector in the inventive embodiments. Any data processing method known in the art for processing of multiple energies CT data may he used. More specifically, multiple energy CT data acquired according to the invention may be used to determine effective atomic number of material within the scanned subject. Material composition of the scanned subject may be analyzed from the multiple energy images or from the pre-processed data.

Exemplary embodiments are described with two groups of detector elements with different spectral response. However, some embodiments may include more than two groups of detector elements, each with associated spectral response, with appropriate modifications to the embodiments described herein. Embodiments of the invention may be used to generate dual energy CT data or to generate CT data for more than two average X-Ray energies.

As was noted in the background, the X-ray detector may rotate with the X-Ray source, or may be stationary. With a stationary detector, the X-Ray source needs to rotate about the scanned subject. However, rotation of the source may be in a form of mechanically rotating an X-Ray tube, or rotating an electron beam such that the X-Ray location of radiation source rotates about the patient or by using a plurality of radiation source around the patient and activating them in sequence. Thus, the term “an X-Ray source adapted to rotate about the subject” should be viewed as generally creating X-Rays from a plurality of locations around the patient in a sequence.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

As used herein, the term “controller” “computer” or “module” may include any processor-based, DSP-based, CPU-based or microprocessor-based system including systems using micro-controllers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects.

Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed:

1. A CT scanner for multiple energy CT scanning of a subject comprising:
an X-Ray source configured to rotate about the subject;
a detector array having a plurality of detector elements, said detector array is configured to acquire attenuation data for X-Rays that have been attenuated by the subject disposed between said X-Ray source and said detector array, said detector array comprising:
at least one region of detector elements having a first spectral response; and
at least one region of detector elements having a second spectral response; and
a controller configured to axially increment the position of the subject respective to said X-Ray source and said detector array such that at least some voxels in the subject that were on lines from said X-Ray source to said detector elements having first spectral response move to lines from said X-Ray source to said detector elements having second spectral response.

2. The CT scanner of claim 1, wherein the scanner is configured to generate images based on data associated with at least said first and said second X-Ray energy spectra.

3. The CT scanner of claim 1, wherein said detector array is an array of detector elements arranged in rows, said rows are substantially parallel to the rotation plane of said source.

4. The CT scanner of claim 2, wherein alternate rows of detector elements have said first and second spectral response.

5. The CT scanner of claim 1, wherein said detector elements in said regions of first and second spectral responses have different X-Ray absorption efficiency.

6. The CT scanner of claim 1, wherein at least one said detector region of a spectral response is shaded from the X-Ray source by partially absorbing X-Ray filters, and wherein at least one detector region of a different spectral response is not shaded from the X-Ray source by said X-Ray filters.

7. The CT scanner of claim 5, wherein said X-Ray filters are arranged in strips facing said detector elements.

8. The CT scanner of claim 1, wherein:
data is acquired while the subject is at a first position;
the subject is axially incremented relative to said X-Ray source and detector array to a second position; and
additional data is acquired while the subject is at a said second position.

9. The CT scanner of claim 1, wherein data is acquired while the subject is being moved relative to said X-Ray source and detector array.

10. The CT scanner of claim 1, wherein said detector array comprises two regions of different spectral sensitivities and wherein the CT scanner is adapted to acquire subject attenuation data and reconstruct images associated with two X-Ray energy spectra.

11. The CT scanner of claim 1, wherein said detector array comprises at least three regions of different spectral sensitivities and wherein the CT scanner is adapted to acquire subject attenuation data and reconstruct images associated with at least three X-Ray energy spectra.

12. The CT scanner of claim 1, wherein the subject is a human patient.

13. The CT scanner of claim 1, wherein the subject is luggage.

14. The CT scanner of claim 1, wherein attenuation data associated with multiplicity of X-Ray energy spectra are used to determine effective atomic number of material within said subject.

15. A CT scanner for multiple energy CT scanning of a subject comprising:
an X-Ray source configured to rotate about the subject;
a detector array configured to acquire attenuation data for X-Rays that have been attenuated by the subject disposed between said X-Ray source and said detector array, said detector array comprising at least one region of a first spectral response: and
at least one region of a second spectral response.

16. The CT scanner of claim 15, and comprising a controller capable of axially shifting the position of the focal spot respective to said scanned subject and said detector array such that at least some voxels in the subject that were on lines from said X-Ray source to said detector elements having first spectral response move to lines from said X-Ray source to said detector elements having second spectral response.

17. The CT scanner of claim 15, and comprising a controller capable of estimating for a detector region of a first spectral response the attenuation data that would have been measured had this region have a second spectral response.

18. The CT scanner of claim 15, and comprising a controller capable of iteratively reconstructing at least one image associated with at least one of said first spectral response and said second spectral response.

19. The CT scanner of claim 15, wherein said detector array is an array of detector elements arranged in rows, said rows arranged in planes parallel to rotation plane of said X-Ray source.

20. The CT scanner of claim 19, wherein said rows of detector elements comprise at least a first section with detector elements of a first spectral response and at least a second section with detector elements of a second spectral response.

21. The CT scanner of claim 20, wherein detector elements of a first spectral response are arranged symmetrically to detector elements of a second spectral response respective a plan passing through a focal spot of said X ray source and the rotation axis.

22. The CT scanner of claim 19, wherein the CT scanner is adapted to reconstruct images of multiple energy spectra from attenuation data received during at least 360° rotation of said X-Ray source.

23. The CT scanner of claim 15, wherein the CT scanner is adapted to scan said subject by succession of rotational scan each at a fixed subject position.

24. The CT scanner of claim 15, wherein the CT scanner is adapted to scan said subject by spiral scan.

25. The CT scanner of claim 15, wherein the detector area having a first spectral response is different than the detector area having a second spectral response.

26. The CT scanner of claim 25, wherein the detector area of a first spectral response is larger by a factor of at least four than the detector area of a second spectral response.

27. The CT scanner of claim 26, wherein the detector area of a first spectral response is larger by a factor of at least ten than the detector area of a second spectral response.

28. The CT scanner of claim 15, wherein the CT scanner is capable of generating images of a first spatial resolution for a first energy spectrum and images of a second spatial resolution for a second energy spectrum.

29. The CT scanner of claim 15, wherein said detector regions of first and second spectral responses are associated with detector elements of different spectral sensitivities.

30. The CT scanner of claim 15, wherein said detector regions of first and second spectral responses are associated with detector elements of different efficiency.

31. The CT scanner of claim 15, wherein at least some of said detector elements belonging to said regions of first and second spectral responses are elements having different dimensions.

32. The CT scanner of claim 15, wherein said detector regions of first and second spectral responses are associated with different X-Ray beam filtering.

33. The CT scanner of claim 15, wherein said detector regions of first and second spectral responses are associated with detectors of different spectral sensitivities and different beam filtering.
